(19) United States
(12) Patent Application Publication
(10) Pub. No.: US 2025/0138010 A1
(43) Pub. Date: May 1, 2025

(54) SPATIAL BARCODING FOR SUSPENSION MASS CYTOMETRY

(71) Applicant: Standard BioTools Canada Inc., Markham (CA)

(72) Inventor: Colin Thom, Palo Alto, CA (US)

(73) Assignee: Standard BioTools Canada Inc., Markham, ON (CA)

(21) Appl. No.: 18/692,892

(22) PCT Filed: Sep. 22, 2022
(86) PCT No.: PCT/US2022/044407

§ 371 Date: Mar. 18, 2024

Related U.S. Application Data
(60) Provisional application No. 63/247,611, filed on Sep. 23, 2021.

(51) Int. Cl. G01N 33/569; G01N 33/53; G01N 33/68

(52) U.S. Cl. CPC G01N 33/56966; G01N 33/5308; G01N 33/6851; G01N 2458/15

(57) ABSTRACT

Aspects of the subject application include applying a spatial barcode to a cellular sample and then performing suspension mass cytometry on the cellular sample. For example, a sample barcode comprising a known distribution of sample barcode isotopes may be applied to the sample such that cells in different locations receive a unique combination or ratio of barcode isotopes, after which cells are suspended (e.g., dissociated from tissue) and processed by suspension mass cytometry. While barcode isotopes are described in a number of embodiments herein, non-enriched elements may be used instead of, or in addition to, enriched isotopes. Mass cytometry methods and reagents are discussed below, followed by a further description of spatial barcoding and kits thereof.

### CROSS-REFERENCES TO RELATED APPLICATIONS

This application claims the benefit of, and priority to U.S. Provisional Application Ser. No. 63/247,611, filed Sep. 23, 2021, which is incorporated here by reference.

### BACKGROUND

Mass cytometry, including imaging mass cytometry (IMC) and suspension mass cytometry (SMC), enables highly multiplexed detection of target analytes through detection of mass tags by mass spectrometry. Mass tags are typically associated with target analytes through an affinity reagent such as an antibody. Mass tags may have one or more copies of a labeling atom (e.g., a single isotope, such as an enriched isotope) that is distinguished from the mass labeling atoms of other mass tags.

Unlike IMC, SMC does not preserve spatial information about the cells analyzed, it has a number of advantages over IMC. For example, a greater number of mass tags have been validated for SMC over IMC, allowing for greater plexity of detection. Further, the analysis of whole cells in SMC allows for better sensitivity to low expressed targets. Finally, SMC enables the analysis of hundreds of cells as cells are rapidly introduced in suspension while the raster scanning of IMC may limit throughput to a few cells per second.

### TECHNICAL FIELD

The field of the subject application relates to spatial barcoding of a sample for analysis by suspension mass cytometry.

### BRIEF DESCRIPTION OF THE FIGURES

FIG. 1 shows a suspension mass cytometry (SMC) workflow in which a suspension of cells are stained with mass tagged antibodies and then analyzed by ICP-MS.

FIG. 2 shows an imaging mass cytometry workflow in which a solid tissue section is stained with mass tagged antibodies and then analyzed by LA-ICP-MS such that spatial information is preserved.

FIG. 3 shows an exemplary spatial barcode of the subject application in which a distribution of barcode isotopes can be used to label the spatial locations of cells.

FIG. 4 shows the different combinations and amounts of the barcode isotopes of cells at different positions along the axis shown in FIG. 3.

FIG. 5 shows a method of spatially barcoding a cellular sample in order to preserve spatial information for analysis by SMC.

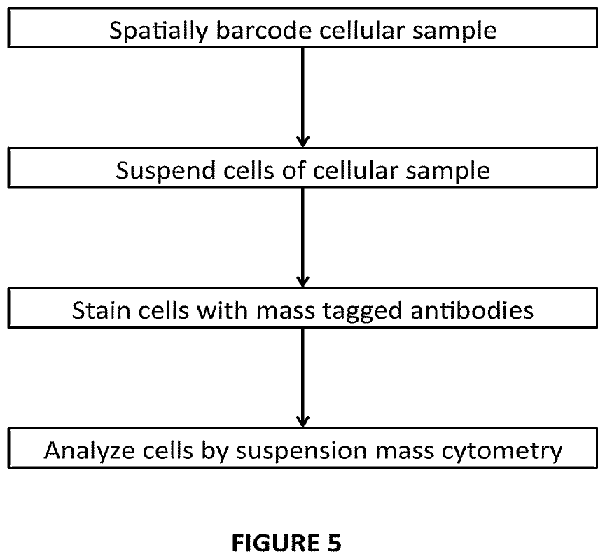

### SUMMARY

Aspects of the subject application include applying a spatial barcode to a cellular sample and then performing suspension mass cytometry on the cellular sample. For example, a sample barcode comprising a known distribution of sample barcode isotopes may be applied to the sample such that cells in different locations receive a unique combination or ratio of barcode isotopes, after which cells are suspended (e.g., dissociated from tissue) and processed by suspension mass cytometry. Aspects of the subject application include:

1. A method of spatial barcoding for suspension mass cytometry, comprising:
a) applying a spatial barcode to a cellular sample such that cells in different locations of the cellular sample are labeled with different combinations or ratios of isotopes, wherein the spatial barcode comprises enriched isotopes having an atomic mass greater than 80 amu;
b) suspending spatially barcoded cells of the cellular sample;
c) staining the suspended cells with mass-tagged affinity reagents, wherein the mass-tagged affinity reagents comprise enriched isotopes having an atomic mass greater than 80 amu and distinct from the atomic mass of the enriched isotopes of the spatial barcode; and
d) analyzing the cells by suspension mass cytometry such that the enriched isotopes of the spatial barcodes and the enriched isotopes of the mass-tagged antibodies are detected on a cell-by-cell basis.

2. The method of aspect 1, wherein the cellular sample is a tissue section.

3. The method of aspect 2, wherein the tissue section has a thickness of greater than 20 microns.

4. The method of aspect 2, wherein the tissue section is a formalin-fixed paraffin-embedded (FFPE) tissue section.

5. The method of aspect 1, wherein the cellular sample is a live solid tissue sample.

6. The method of aspect 1, wherein the cellular sample is a fresh-frozen solid tissue sample.

7. The method of aspect 1, wherein the cellular sample is an organoid.

8. The method of aspect 1, wherein the cellular sample is a solid tissue biopsy.

9. The method of aspect 1, wherein the cellular sample is embedded in a protein matrix.

10. The method of any one of aspects 1 to 9, further comprising applying a metal-containing biosensor or metal-containing histochemical compound to the cellular sample prior to the step of suspending the spatially barcoded cells.

11. The method of any one of aspects 1 to 10, wherein the cells at different locations comprise different combination of isotopes.

12. The method of aspect of any one of aspects 1 to 11, wherein the cells at different locations comprise different ratios of isotopes.

13. The method of any one of aspects 1 to 12, wherein the spatial barcode is a solid support comprising a distribution of spatial barcode isotopes, wherein the spatial barcode isotopes are patterned across at least a portion of the solid support such that each location is uniquely barcoded.

14. The method of aspect 13, wherein the solid support is a film or matrix.

15. The method of any one of aspects 1 to 12, wherein the device comprises a microfluidic device.

16. The method of aspect 15, wherein the microfluidic device comprises channels configured to deliver a different combination or ratio of barcode isotopes to different locations of a sample.

17. The method of any one of aspects 1 to 16, wherein a first barcode isotope increases along a first spatial dimension in the cellular sample.

18. The method of aspect 17, wherein a second barcode isotope increases along a second spatial dimension in the cellular sample.

19. The method of aspect 18, wherein a third barcode isotope is spotted at multiple locations in the cellular sample.

20. The method of any one of aspects 1 to 16, wherein at least 2 barcode isotopes have a unique spatial distribution.

21. The method of any one of aspects 1 to 20, wherein a normalization barcode isotope is evenly distributed across at least a portion of the cellular sample.

22. The method of any one of aspects 1 to 21, wherein the spatial barcode is arranged on a solid support that is applied to the cellular sample.

23. The method of any one of aspects 1 to 21, wherein the spatial barcode is applied to the cellular sample by a microfluidic device.

24. The method of any one of aspects 1 to 21, wherein applying the spatial barcode comprises diffusing at least one isotope across the cellular sample.

25. The method of any one of aspects 1 to 21, wherein applying the spatial barcode comprises diffusing the spatial barcode isotopes across the cellular sample in different directions.

26. The method of any one of aspects 1 to 25, wherein applying the spatial barcode comprises contacting the cellular sample with a solid support and then applying a solution of one or more spatial barcode isotopes to the opposite side of the solid support from the cellular sample.

27. The method of aspect 26, wherein the solid support is a gel or a porous substrate that provides a gradient of permeability.

28. The method of any one of aspects 1 to 22, wherein the spatial barcode comprises a solid support spotted with different combinations or ratios of barcode isotopes.

29. The method of any one of aspects 1 to 28, further comprising applying an even distribution of a normalization isotope to the cellular sample before and/or after the step of suspending the cells.

30. The method of any one of aspects 1 to 29, wherein the spatial barcode comprises a thiol-reactive moiety.

31. The method of any one of aspects 1 to 30, further comprising stopping binding of the spatial barcode to the cellular sample by one or more of adjusting pH, adjusting temperature, or adding a reactive moiety in excess in solution.

32. The method of aspect 31, wherein the reactive moiety is a thiol that reacts with the spatial barcode.

33. The method of aspect 32, wherein the reactive moiety is conjugated to a steric group that prevents entry of the reactive moiety into the cellular sample.

34. The method of any one of aspects 1 to 33, wherein the spatial barcode comprises a dye or fluorophore that indicates the distribution of the spatial barcode isotopes.

35. The method of any one of aspects 1 to 34, further comprising enriching cells after the step of dissociating cells and prior to the step of analyzing the cells.

36. The method of any one of aspects 1 to 35, wherein enriching comprises enriching cells of one or more types based on protein expression.

37. The method of any one of aspects 1 to 36, further comprising applying a sample barcode to the suspended cells of the cellular sample and combining the suspended cells with cells comprising a different sample barcode prior to the step of staining the suspended cells.

38. The method of aspect 37, further comprising excluding doublets comprising two different sample barcodes from a dataset obtained from the step of analyzing.

39. The method of any one of aspects 1 to 38, wherein analyzing the cells by suspension mass cytometry comprises introducing the cells to ICP-MS.

40. The method of aspect 39, wherein analyzing the cells by suspension mass cytometry comprises introducing the cells to ICP-TOF-MS.

41. The method of any one of aspects 1 to 40, wherein suspending the cells comprises enzymatically treating the cells.

42. The method of any one of aspects 1 to 41, wherein suspending the cells comprises applying a shear force to the cells.

43. The method of any one of aspects 1 to 42, further comprising assigning spatial coordinates to cells based on the enriched isotopes of the spatial barcodes detected for individual cells.

44. The method of any one of aspects 1 to 43, further comprising calculating the proximity of cells to one another based on the enriched isotopes of the spatial barcodes detected for individual cells

45. The method of any one of aspects 1 to 44, further comprising analyzing the 3D spatial distribution of cells based on the enriched isotopes of the spatial barcodes detected for individual cells

46. The method of any one of aspects 1 to 45, further comprising optically imaging the cellular sample prior to the step of suspending cells of the cellular sample.

47. The method of aspect 46, wherein imaging comprises imaging fiducials indicating the distribution of isotopes of the spatial barcode.

48. The method of aspect 46 or 47, wherein imaging comprises imaging a serial section of the cellular sample.

49. The method of any one of aspects 46 to 48, wherein the imaging comprises imaging a histochemical stain of the cellular sample.

50. The method of any one of aspects 46 to 49, further comprising defining a region of interest in the image and identifying cells in the region of interest based on the spatial barcodes of cells detected by suspension mass cytometry.

51. The method of aspect 50, further comprising analyzing spatial relationships of cells identified in the region of interest.

52. A spatial barcode kit for suspension mass cytometry, comprising:
a plurality of barcodes comprising enriched isotopes having an atomic mass greater than 80 amu; and
a device configured to apply the spatial barcodes to a cellular sample in a spatially arranged manner.

53. The kit of aspect 52, wherein the device comprises a solid support, wherein the enriched isotopes are patterned across at least a portion of the solid support such that each location is uniquely barcoded.

54. The kit of aspect 53, wherein the solid support comprises a film.

55. The kit of aspect 54, wherein the barcodes are adsorbed on the surface of the film.

56. The kit of aspect 54, wherein the barcodes are spotted onto the solid support, optionally wherein the barcodes are dried onto the solid support.

57. The kit of aspect 52, wherein the solid support comprises a gel.

58. The kit of aspect 52, wherein the solid support is a gel or a porous substrate that provides a gradient of permeability.

59. The kit of aspect 52, wherein the solid support is a gel comprising a gradient of density and/or thickness.

60. The kit of aspect 52, wherein the device comprises a microfluidic device.

61. The kit of aspect 60, wherein the microfluidic device comprises channels configured to deliver a different combination or ration of barcodes to different locations of a sample.

62. The kit of any one of aspects 52 to 61, wherein barcodes comprising different enriched isotopes or combinations or ratios thereof are packaged separately.

63. The kit of any one of aspects 52 to 62, wherein the barcodes are packaged in unique combinations of enriched isotopes.

64. The kit of any one of aspects 52 to 62, wherein the barcodes are packaged in unique ratios of enriched isotopes.

65. The kit of any one of aspects 52 to 64, wherein the barcodes are suspended in an organic solvent.

66. The kit of aspect 65, wherein the organic solvent is DMSO.

67. The kit of any one of aspects 52 to 66, wherein the barcodes comprise small molecule barcodes.

68. The kit of aspect 67, wherein the small molecule barcodes have a molecular weight of less than 500 daltons.

69. The kit of aspect 67, wherein the small molecule barcodes comprise cisplatin or a derivative thereof.

70. The kit of any one of aspects 65 to 69, further comprising a stop reagent that reacts with the barcodes.

71. The kit of any one of aspects 65 to 70, wherein the barcodes comprise a cell binding moiety and a barcode binding moiety; wherein the barcode binding moiety selectively binds to a moiety on the barcodes.

72. The kit of aspect 71, wherein the cell binding moiety is a lipid that embeds in the cell membrane.

73. The kit of aspect 71, wherein the cell binding moiety is an antibody that binds to proteins on the cell surface.

74. The kit of aspect 71, wherein the barcode binding moiety is an avidin or biotin.

75. The kit of aspect 74, wherein the barcode binding moiety is an oligonucleotide sequence complimentary to at least 10 nucleotides of a oligonucleotide sequence of the barcodes. 76. The kit of aspect wherein the barcode binding moiety is a strain-promoted click chemistry group.

77. The kit of any one of aspects 52 to 70, wherein the barcode comprises a thiol-reactive moiety.

78. The kit of any one of aspects 52 to 70, wherein the barcode comprises an amine-reactive moiety 79. The kit of any one of aspects 52 to 78, wherein the barcodes comprise a maleimide-functionalized organometal.

80. The kit of aspect 79, wherein the organometal is organotellurium.

80. The method of any one of aspects 1 to 51 using the kit of any one of aspects 52 to 80.

82. A spatially barcoded cellular sample for suspension mass cytometry, wherein the spatial barcode comprises enriched isotopes having an atomic mass greater than 80 amu.

### DESCRIPTION

Aspects of the subject application include applying a spatial barcode to a cellular sample and then performing suspension mass cytometry on the cellular sample. For example, a sample barcode comprising a known distribution of sample barcode isotopes may be applied to the sample such that cells in different locations receive a unique combination or ratio of barcode isotopes, after which cells are suspended (e.g., dissociated from tissue) and processed by suspension mass cytometry. While barcode isotopes are described in a number of embodiments herein, non-enriched elements may be used instead of, or in addition to, enriched isotopes. Mass cytometry methods and reagents are discussed below, followed by a further description of spatial barcoding and kits thereof.

### Mass Cytometry

As used herein, mass cytometry is any method of detecting mass tags in individual cells of a cellular sample, such as by simultaneously detecting a plurality of distinguishable mass tags with single cell resolution. Mass cytometry includes suspension mass cytometry and imaging mass cytometry (IMC). Mass cytometry may atomize and ionize mass tags of a cellular sample by one or more of laser radiation, ion beam radiation, electron beam radiation, and/or inductively coupled plasma (ICP). Mass cytometry may simultaneously detect distinct mass tags from single cells, such as by time of flight (TOF) or magnetic sector mass spectrometry (MS). Examples of mass cytometry include suspension mass cytometry where cells are flowed into and ICP-MS and imaging mass cytometry where a cellular sample (e.g., tissue section) is sampled, for example by laser ablation (LA-ICP-MS) or by a primary ion beam (e.g., for SIMS).

Mass tags may be sampled, atomized and ionized prior to elemental analysis. For example, mass tags in a biological sample may be sampled, atomized and/or ionized by radiation such as a laser beam, ion beam or electron beam. Alternatively or in addition, mass tags may be atomized and ionized by a plasma, such as an inductively coupled plasma (ICP). In suspension mass cytometry, whole cells including mass tags may be flowed into an ICP-MS, such as an ICP-TOF-MS. In imaging mass cytometry, a form of radiation may remove (and optionally ionize and atomize) portion (e.g., pixels, region of interest) of a solid biological sample, such as a tissue sample, including mass tags. Examples of IMC include LA-ICP-MS and SIMS-MS of mass tagged sample. In certain aspects, ion optics may deplete ions other than the isotope of the mass tags. For example, ion optics may remove lighter ions (e.g., C, N, O), organic molecular ions. In ICP applications, ion optics may remove gas such as Ar and/or Xe, such as through a high-pass quadrupole filter. In certain aspects, IMC may provide an image of mass tags (e.g., targets associated with mass tags) with cellular or subcellular resolution.

Similar to fluorescent immunohistochemistry methods, mass cytometry (including imaging mass cytometry) workflows may include cell (e.g., tissue) fixation and/or permeabilization prior to staining with antibodies and/or other affinity reagents. In contrast to fluorescent methods, in mass cytometry mass tags (e.g., comprising heavy metals not endogenous to the cell) are associated with target analytes through affinity reagents such as antibodies. Imaging mass cytometry, like fluorescent microscopy, may include an antigen retrieval step where the sample is exposed to conditions such as heat to expose target analytes for binding by affinity reagents. Unbound affinity reagents are typically washed off before detection of mass tags by mass spectrometry. Of note, other methods of detection such as elemental analysis (e.g, emission spectroscopy or X-ray dispersion spectroscopy) are also within the scope of the subject application.

Additional reagents for mass cytometry include metal-containing biosensor(s) (e.g., that is deposited or bound under conditions such as hypoxia, protein synthesis, cell cycle and/or cell death) and/or metal containing histochemical compound(s) that bind to structures (e.g., DNA, cell membrane, strata) based on chemical properties. Such additional reagents may be applied prior to suspending the cells form a solid cellular sample or applied to suspended cells that have already been spatially barcoded. In addition, mass tags (e.g., of the subject application or other mass tags) may be combined to provide a unique sample barcode, so as to label a particular sample or experimental condition prior to pooling with other samples or experimental conditions.

Cells in biological samples as discussed herein may be prepared for analysis of RNA and/or protein content using the methods and apparatus described herein. In certain aspects, cells are fixed and permeabilized prior to the hybridization step. Cells may be provided as fixed and/or permeabilized. Cells may be fixed by a crosslinking fixative, such as formaldehyde, glutaraldehyde. Alternatively or in addition, cells may be fixed using a precipitating fixative, such as ethanol, methanol or acetone. Cells may be permeabilized by a detergent, such as polyethylene glycol (e.g., Triton X-100), Polyoxyethylene (20) sorbitan monolaurate (Tween-20), Saponin (a group of amphipathic glycosides), or chemicals such as methanol or acetone. In certain cases, fixation and permeabilization may be performed with the same reagent or set of reagents. Fixation and permeabilization techniques are discussed by Jamur et al. in “Permeabilization of Cell Membranes” (Methods Mol. Biol., 2010).

An inductively coupled plasma (ICP) is a type of plasma source in which the energy is supplied by electric currents which are produced by electromagnetic induction (i.e., by time-varying magnetic fields). Industrial scale applications of ICP include micromachining (e.g., etching or cleaning) or waste disposal. Such applications may not generate plasma in an ICP torch, may not use an ICP load coil, may not operate under atmospheric conditions, and/or may not be at a scale suitable for atomic analysis of a sample (e.g., the plasma generated may be at least an order of magnitude larger that that of ICP analyzers). As such, the physics of industrial ICP is different than for ICP analysis using an ICP torch, and may be outside the scope of aspects of the present disclosure. Discussed herein are systems and methods using ICP torches, such as ICP analyzers.

An overview of ICP mass spectrometers (ICP-MS) is provided in Montaser, Akbar, ed. Inductively coupled plasma mass spectrometry. John Wiley & Sons, 1998, which includes a description of vortex flow and ignition. Sample introduction and ICP torch considerations is similar for atomic emission spectroscopy (AES), also known as optical emission spectroscopy, which is also within the scope of the subject application. Atomic spectroscopy, as used herein, is identical to atomic analysis and may include atomic mass spectrometry (such as ICP-MS) or ICP-AES. Suitable samples include biological samples, geological samples, and articles of manufacture. In certain aspects, a biological sample may be a fluid comprising biomolecules and/or contaminants (e.g., metal toxins), or particles such as cell (e.g., in suspension or in a tissue section) or beads (e.g., used to assay biomolecules).

Aspects of the subject application include ICP-torch systems and methods for mass cytometry, which is the detection of mass tags in cells or beads by mass spectrometry. Mass cytometry is discussed in US patent publications US20050218319, US20160195466, and US20190317082, which are incorporated by reference in their entirety. Mass cytometry may be of suspended particles (e.g., cells or beads), or of particles produced from a solid sample, such as laser ablation plumes produced from a tissue section. In suspension mass cytometry, a suspension of cells or beads comprising mass tags are analyzed by atomic mass spectrometry. Imaging mass cytometry by laser ablation (LA) ICP-MS is described in US patent publications US20160056031 and US20140287953, which are incorporated herein by reference. Imaging mass cytometry by LA-ICP-MS is also described by Giesen, Charlotte, et al. in “Highly multiplexed imaging of tumor tissues with subcellular resolution by mass cytometry.” (Nature methods 11.4 (2014): 417-422).

Mass tags may be metal tags bound to affinity reagents (e.g., antibodies, oligonucleotides, avidin, or other biomolecules that specifically bind a target biomolecule). For example, metal nanoparticles or metal-chelating polymers may be attached (e.g., covalently bound) to affinity reagents, which are then applied to the sample. Suitable mas tags are described in US patent publications US20040072250 and US20080003616, which are incorporated by reference in their entirety. In certain aspects, some mass tags are not coupled to affinity reagents, such as metal containing drugs or histochemical stains.

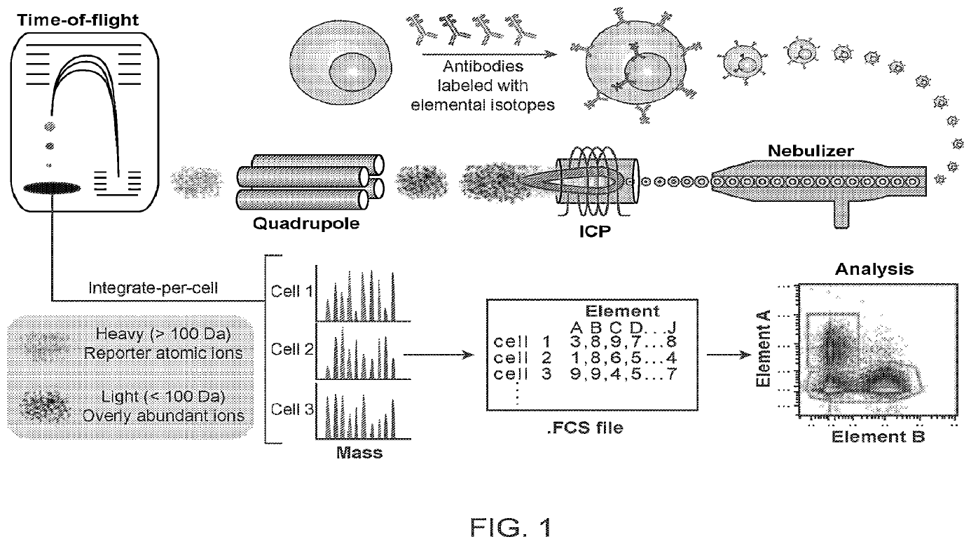

In suspension mass cytometry (SMC) a suspension of cells are stained with mass-tagged affinity reagents (e.g., antibodies) and analyzed by mass spectrometry. In certain aspects, stained cells are flowed into an ICP-MS system in which the cells are atomize and ionized followed by simultaneous analysis of mass tags such as by time-of-flight mass spectrometer as shown in FIG. 1 or magnetic sector mass spectrometry. Due to the presence of argon dimer in the plasma, mass tags having enriched isotopes with an atomic mass over 80 may be used and lighter ions may be filtered out by ion optics. Mass cytometry systems, including exemplary suspension mass cytometry systems, are described in US patent publication number US20120056086, which is incorporated herein by reference. The CyTOF system is a mass cytometer commercially available from Fluidigm and uses ICP-TOF-MS as depicted in FIG. 1.

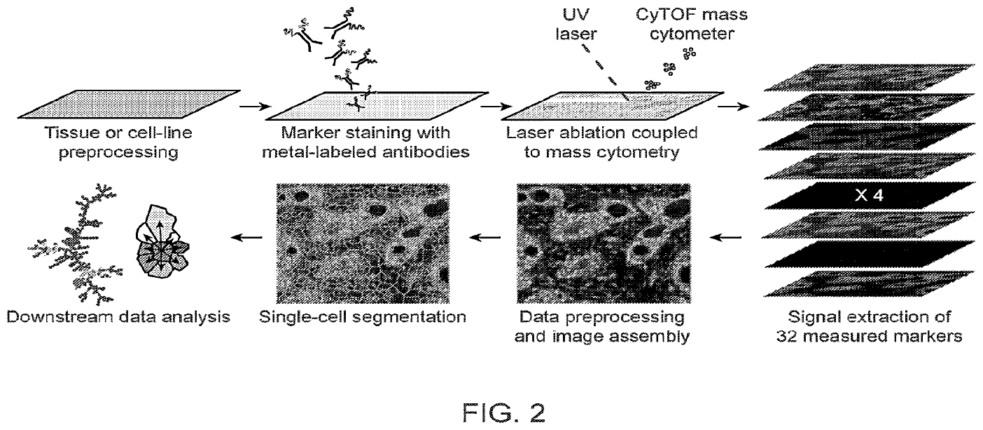

In IMC, a tissue sample may be a section e.g. having a thickness within the range of 1-10 μm, such as between 2-6 μm may be used. In some cases, an ultrathin section less than 500 nm, 200 nm, 100 nm or 50 nm thick may be used, such as sample cut from a resin-embeded tissue block. Techniques for preparing such sections are well known from the field of IHC e.g. using microtomes, including dehydration steps, fixation, embedding, permeabilization, sectioning etc. Thus, a tissue may be chemically fixed and then sections can be prepared in the desired plane. Cryosectioning or laser capture microdissection can also be used for preparing tissue samples. Samples may be permeabilised e.g. to permit of reagents for labelling of intracellular targets. Even after antigen retrieval (e.g., by heating), access to an analyte by an affinity reagent may be sterically hindered. As such, smaller affinity reagents and certain mass tags may best allow for the affinity reagent to access its target analyte. IMC may be performed by laser ablation ICP-MS, such as shown in FIG. 2.

Unlike IMC, SMC does not preserve spatial information about the cells analyzed, it has a number of advantages over IMC. For example, a greater number of mass tags have been validated for SMC over IMC, allowing for greater plexity of detection. Further, the analysis of whole cells in SMC allows for better sensitivity to low expressed targets. Finally, SMC enables the analysis of hundreds of cells as cells are rapidly introduced in suspension while the raster scanning of IMC may limit throughput to a few cells per second.

### Mass Tags

Mass tags may be metal tags bound to affinity reagents (e.g., antibodies, oligonucleotides, avidin, or other biomolecules that specifically bind a target biomolecule). For example, metal nanoparticles or metal-chelating polymers may be attached (e.g., covalently bound) to affinity reagents, which are then applied to the sample (e.g., cellular sample). Suitable mas tags are described in US patent publications US20040072250 and US20080003616, which are incorporated by reference in their entirety. In certain aspects, some mass tags are not coupled to affinity reagents, such as metal containing drugs or histochemical stains. Mass tags may comprise an enriched isotope, such as an enriched isotope above 80 amu that can be detected separate from endogenous elements from a cellular sample and/or argon dimer from an ICP.

As used herein in the context of mass cytometry, signal amplification is the association of more than 30, more than 50, more than 100, more than 200, or more than 500 labeling atoms (e.g., of an enriched isotope) with a target analyte (i.e., a single instance of the target analytes bound by a affinity reagent). In certain aspects, labeling atoms may be heavy metals, such as lanthanides or transition metals. In certain aspects, signal amplification may be performed for more than 2, 5, 10 or 20 target analytes. In certain aspects, signal amplification may include use of branched conjugation of a mass tag to affinity reagent, a high sensitivity polymer, a large mass tag particle, a mass tag nanoparticle, and/or a hybridization scheme. In certain aspects, signal amplification uses a mass tag polymer.

As described herein, signal amplification may be by use of mass tags comprising a high number of labeling atoms and/or by association of a larger number of mass tags with a single target analyte (such as through hybridization based signal amplification and/or conjugation of mass tags to affinity reagents through branched heterofunctional linkers). In certain aspects, a single mass tag may have more than 30, 50, 100, 200, 500, or 1000 labeling atoms. In certain aspects, the hydrodynamic diameter of a mass tag may be low, such as less than 20 nm, less than 15 nm, less than 10 nm, less than 5 nm, less than 3 nm, or less than 2 nm. The hydrodynamic diameter may be less than 1000 nm³, less than 500 nm³, less than 100 nm³, less than 50 nm³, less than 20 nm³, or less than 10 nm³. Techniques such as EM may be used to identify the size, and light scattering may be used to identify the hydrodynamic diameter of mass tags, such as larger mass tags described herein. Further, chromatography methods including as size exclusion and ion exchange (e.g., anion-exchange) chromatography may be used to characterize mass tags, such as smaller mass tags described herein.

A variety of suitable conjugation means are known in the art. For example, a mass tag may be conjugated to a biologically active material, such as through covalent binding (e.g., amine chemistry, thiol chemistry, phosphate chemistry, an enzymatic reaction, a redox reaction (such as with a metal halide), and affinity intermediate (e.g., streptavidin or biotin), or a form of click chemistry such as strain promoted click chemistry or metal-catalyzed click chemistry). In certain aspects, the conjugation methods described herein may be used to conjugate an oligonucleotide to an affinity reagent, such as when a hybridization scheme is used to indirectly associate mass tagged oligonucleotides with an affinity reagent-oligonucleotide conjugate.

A mass tag may be conjugated to a biologically active material, such as through covalent binding (e.g., amine chemistry, thiol chemistry, phosphate chemistry, an enzymatic reaction, or a form of click chemistry such as strain promoted click chemistry or metal-catalyzed click chemistry). The biologically active material may be an affinity reagent (such as an antibody or fragment thereof, aptamer, lectin, and so forth) or an oligonucleotide probe that hybridizes to an endogenous target (e.g., DNA or RNA) or an intermediate (e.g., antibody-oligonucleotide intermediate and/or a hybridization scheme of oligonucleotides). As described herein, suitable attachment chemistries may include carboxyl-to-amine reactive chemistry (e.g., such as reaction with carbodiimide), amine-reactive chemistry (e.g., such as reaction with NHS ester, imidoester, pentafluorophenyl ester, hydroxymethyl phosphine, etc.), sulfhydryl reactive chemistry (e.g., such as reaction with maleimide, haloacetyl (Bromo- or Iodo-), pyridyldisulfide, thiosulfonate, vinylsulfone, etc.), aldehyde reactive chemistry (e.g., such as reaction with hydrazide, alkoxyamine, etc.), hydroxyl reactive chemistry (e.g., such as reaction with isothiocyanate). Alternative method of attachment include click chemistry, such as strain promoted click chemistry (such as by DBCO-azide or TCO-tetrazine).

The polymer may be functionalized to bind a biologically active material. In certain aspects, the polymer may be functionalized through thiol reactive chemistry, amine reactive chemistry or click chemistry. For example, the polymer may be functionalized for thiol reactivity (e.g., via a maleimide group to attach to thiol groups on the Fc portion of an antibody that is reduced, e.g., by TCEP reduction). The type of conjugation, and conjugation conditions (e.g., concentration of a reducing agent) may be different based on the type of affinity reagent to maintain integrity of the affinity reagent.

For example, a polymer mass tag (e.g., comprising a plurality of metal binding groups, such as metal chelating pendant groups) may be functionalized with a thiol reactive group such as maleimide. In certain aspects, an affinity reagent may comprise cysteines that may be reduced (e.g., by TCEP reduction) to provide thiols for conjugation to the polymer. However, the cysteines on the affinity reagent may not be accessible, disruption of the cysteine may reduce the affinity of the affinity reagent, or the reduction step may reduce the affinity of the affinity reagent. In such cases, other functional groups on the affinity reagent may be thiolated prior to conjugation, even on an affinity reagent that already comprises thiols or cysteines. For example, a recombinant antibody may be designed to be smaller (e.g. to reduce steric hindrance and thereby improve binding), and may therefore not have an accessible cysteine on the Fc region. In such cases, amines may be indirectly thiolated, such as by reaction with succinimidyl acetylthioacetate followed by removal of the acetyl group with 50 mM hydroxylamine or hydrazine. In anther example, amines can be indirectly thiolated by reaction with succinimidyl 3-(2-pyridyldithio) propionate followed by reduction of the 3-(2-pyridyldithio) propionyl conjugate with DTT or TCEP. Reduction releases the 2-pyridinethione chromophore, which can be used to determine the degree of thiolation. Alternatively, thiols can be incorporated at carboxylic acid groups by an EDAC-mediated reaction with cystamine, followed by reduction of the disulfide with DTT or TCEP. Finally, tryptophan residues in thiol-free proteins can be oxidized to mercaptotryptophan residues, which can then be conjugated to a mass tag comprising an iodoacetamide or maleimide. In certain aspects, the reduction step described for thiolation may be skipped or may be less stringent than would be needed for conjugation to a thiol of a reduced cysteine, such that a maleimide functionalized mass tag polymer is conjugated to the thiolated moiety and not at a reduced cysteine of the affinity reagent. In certain aspects, a non-peptide based affinity reagent (such as an oligonucleotide) may be more resilient, and conjugation may include reduction at a TCEP or DTT concentration at or above 25 mM or at or above 50 mM. In certain aspects, a conjugation of a non-peptide based affinity reagent may include harsher temperatures, such as denaturation through heat or freezing.

In certain aspects, the affinity reagent (such as an oligonucleotide or peptide) may be small, such as within 50% of the size of a polymer mass tag. This may provide better tissue penetration and/or reduced steric hindrance, but may complicate purification of mass tagged affinity reagent in a filtering step. As such, the mass tag may be modified to present an epitope that can allow affinity based purification. In certain aspects,

A variety of different metal-catalyst free click chemistry reactions, such as strain promoted reactions, can be used according to certain aspects of the present disclosure.

Mass tags of the subject application include polymers comprising a plurality of labeling atoms, for example loaded on metal chelating pendant groups or incorporated into a backbone of the polymer. In certain aspects, mass tagged polymers may be provided separately from an elemental or isotopic composition (e.g., that can be loaded onto chelators of the mass tag polymer, or that is already loaded onto chelators of the mass tag polymer). Mass tagged polymers may be provided attached to an affinity reagent such as an antibody or fragment thereof. In certain aspects, a mass tag polymer may have, or may be capable of binding (e.g., through chelation), more than 10, more than 20, more than 30, more than 50, more than 100, or more than 200 labeling atoms (e.g., of a single isotope, such as an enriched isotope). High sensitivity polymers may have, or may be capable of binding (e.g., on average) more than 30, more than 50, more than 100, or more than 200 labeling atoms.

High sensitivity polymers may be linear or branched. A branched polymers may be a dendritic polymer (e.g., comprising at least second, third, or fourth generation branches) or a star polymer (e.g., comprising at least three linear polymers spreading from a central core). In certain aspects, high sensitivity polymers may include solubility enhancing pendant groups (e.g., having polar groups such as PEG) that do not have a chelator, in addition to metal chelating pendant groups.

Chelators as used herein refer to a group of ligands that together coordinate (e.g., stably coordinate) a metal atom. The chelators may be present on pendant groups of the polymer and/or incorporated into the polymer backbone. In certain aspects, the chelators are included in pendant groups of the polymer.

In certain aspects, a polymer may include one or more pendant groups that include a ligand such as hydroxamate (used interchangeable herein with hydroxamic acid), azamacrocycle, phenoxyamine, salophen, cyclam, and/or derivative(s) thereof. The polymer may include a chelator known in the art, or a derivative thereof, that includes hydroxamate, azamacrocycle, phenoxyamine, salophen, or cyclam. In certain aspects, a chelator of the subject application may coordinate six or more, more than six, or eight sites on a zirconium or hafnium atom. For example, a chelator may form an octa-coordinate complex with at least one of zirconium or hafnium. For example, at least one of zirconium and hafnium may form an octa-coordinate complex with pendant groups of the polymer.

In certain aspects, a chelator of a polymer includes hydroxamate groups, such as in DFO and/or a derivative thereof. Alternatively or in addition, the polymer may include azamacrocycles, such as a 1,4,7,10-tetraazacyclododecane-1,4,7,10-tetraacetic acid (DOTA) chelator or a derivative thereof. In certain embodiments, a chelator may include one of DOTAM, DOTP and DOTA (e.g., loaded with or provided separately from a zirconium or hafnium isotope). In certain aspects, a chelator is a DOTA derivative with improved binding of zirconium or hafnium (and potentially reduced binding to a lanthanide) as compared to DOTA. For example, a DOTA derivative may coordinate eight sights on a zirconium and/or hafnium atom, and may optionally include spacing between ligands that assists with binding (e.g., stably binding) zirconium and/or hafnium. For example, the DOTA derivative may have increased binding to zirconium and/or hafnium as compared to a lanthanide isotope.

Metal nanoparticle mass tags, such as nanometer scale metal clusters, provide a high density of labeling atoms but have a number of drawbacks. Functionalization of a nanoparticle with an inert surface for attachment to an affinity reagent is nontrivial, and would usually result in multiple attachment sites for affinity reagents. Synthesis of small nanoparticles (e.g., less than 10 nm or less than 5 nm) may be difficult, resulting in steric hindrance, poor solubility, poor colloidal stability (aggregation), and non-specific binding. Synthesis of metal cluster nanoparticles may be difficult (e.g., may require high temperatures and may be sensitive to synthesis conditions). Nanoparticles may not be uniform in size.

In certain aspects, a metal nanoparticle may be synthesized at moderate temperature (e.g., less than 100 degrees Celsius, less than 50 degrees Celsius, or less than 37 degrees Celsius) in the presence of an stabilizer, such as an organic stabilizer. For example, the metal nanoparticle may be a quantum dot. In certain aspects the organic stabilizer may comprise a thiol group, such as a cysteine.

In certain aspects, the stabilizer may act as a capping agent. The stabilizer may be on a polymer, and the nanoparticle may be synthesized on the polymer. The size of the polymer may limit (e.g., control) the size of the nanoparticle. The particle may include a linear or branched portion presenting multiple instances of the stabilizer. The particle may further include a attachment group for attaching the polymer (including a nanoparticle synthesized on the polymer) to a single affinity reagent. The mass tag may have low polydispersity, such as a polydispersity index of less than 1.5, 1.2, or 1.1. As such, the nanoparticles may be uniform in size (e.g., may have a polydispersity index of less than 1.5, 1.2, or 1.1). The majority of nanoparticles may have a small diameter, such as a diameter between 1 and 10 nm, 1 and 5 nm, 1 and 3 nm, 1 and 2 nm, 2 and 5 nm, 2 and 3 nm. Nanoparticles may be of an element having a plurality of isotopes, such as Cadmium or Tellurium, but may have a non-natural composition of isotopes (such as an enriched isotope of Cadmium or Tellurium). Nanoparticles may be monodisperse. In certain aspects, a polymer may include a plurality of nanoparticles. In certain aspects, the rate of seeding nanoparticle growth on the polymer may be slower than the rate of growth. Rapid growth of the nanoparticle may consume the stabilizing groups on the polymer such that the polymer does not associate with nanoparticles growing on other polymers. Polymers may be dispersed to reduce the rate of multiple polymers associating with the same nanoparticle as it grows. In certain aspects, pre-formed (pre-seeded) nanoparticles may be mixed with polymers prior to growth of the nanoparticle on the polymer. In certain aspects, the polymer may have between 10 and 10000, between 10 and 1000, between 10 and 100, between 10 and 50, between 20 and 500, or between 20 and 100 instances of a stabilizer. In certain aspects, a polymer may have less than 10, or even just a single instance of a stabilizer, and stabilizer present in solution may enable nanoparticle growth on the polymer. The same or different stabilizer to the polymer may be provided in solution during synthesis of the nanoparticle on the polymer.

As described previously, small cadmium (CdSe, CdS, and CdTe) nanoparticles may be formed in the presence of thiol-alcohol or thiol-acid stabilizers. Synthesis of cysteine stabilized monodisperse CdsS nanoparticles may seeded with such nanoparticles. While synthesis and association of gold nanoparticles on large poly (cysteine) polymers has been reported without showing uniform size, monodispersity, or cysteine acting as a stabilizer or capping agent for gold nanoparticles. Of note, the synthesis of these nanoparticles were performed at moderate temperatures.

Aspects include Cd or CdTe nanoparticles comprising enriched isotopes and synthesized on a thiol-presenting polymer, such as a polycysteine polymer, and the use of such nanoparticles as mass tags for affinity reagents. In certain aspects affinity reagent itself may provide the stabilizing agent, such as a thiol group (e.g., presented by a reduced antibody), and the nanoparticle may be synthesized directly on the affinity reagent. Provided the thiol group is not proximal to the binding site of the affinity reagent, direct synthesis may keep the nanoparticle from sterically interfering with binding.

### Affinity Reagents and Small Moieties as Affinity Reagents

An affinity reagent may bind its target analyte non-covalently, such as through affinity (e.g., tertiary structure) or hybridization. Certain aspects of the present disclosure also provide a method of labelling a sample (e.g., cellular sample) using a mass tagged affinity reagent of the disclosure, optionally are methods of labelling a sample using multiple such mass tagged affinity reagents, for example wherein the mass-tagged affinity reagents include affinity reagents of different types, for instance an antibody affinity reagent (including multiple antibody affinity reagents), a nucleic acid affinity reagent (including multiple nucleic acid affinity reagents), a lectin (including multiple lectins), a sugar (including multiple sugars) and a DNA intercalator (including multiple DNA intercalators). Similarly, certain aspects of the present disclosure include use of a mass tagged affinity reagent of the disclosure for labelling a sample, such as the use of multiple such mass tagged affinity reagents for labelling a sample, for example wherein the mass-tagged affinity reagents include affinity reagents of different types, for instance an antibody affinity reagent (including multiple antibody affinity reagents), a nucleic acid affinity reagent (including multiple nucleic acid affinity reagents), a lectin (including multiple lectins), a sugar (including multiple sugars) and a DNA intercalator (including multiple DNA intercalators). Accordingly, certain aspects of the present disclosure also provide a sample labelled according to the disclosure, such as a sample labelled with a mass tagged affinity reagent of the disclosure, optionally a sample labelled with multiple such mass tagged affinity reagents, for example wherein the mass-tagged affinity reagents include affinity reagents of different types, for instance an antibody affinity reagent (including multiple antibody affinity reagents), a nucleic acid affinity reagent (including multiple nucleic acid affinity reagents), a lectin (including multiple lectins), a sugar (including multiple sugars) and a DNA intercalator (including multiple DNA intercalators).

In certain aspects, an affinity reagent may be a derivative on an antibody (such as an antibody fragment, nanobody, or synthetic antibody), nucleic acid aptamers, and non-immunoglobulin protein (e.g., avidin), peptides (e.g., matching or derived from binding domains of a protein such as a zinc finger that binds nucleic acids or a receptor binding domain that binds a small peptide or molecule, and so forth) or their corresponding analytes. In such cases, the affinity reagent may be a small moiety, smaller than a traditional antibody. For example, a small moiety affinity reagent may be less than 50, less than 30, less than 20, less than 10, or less than 5 kDa in molecular weight. A small moiety affinity reagent may allow for a larger mass tag without drawbacks discussed herein. A small moiety affinity reagent may better permeate a cell or tissue, for example allowing for deeper staining of a tissue.

Different affinity reagents may be impacted differently by conjugation method and mass tag. As such, aspects of the invention include use of different signal amplification methods for different affinity reagents in analysis of the same sample. The different affinity reagents may be different types of affinity reagents (e.g., oligonucleotides vs antibodies), different antibody isotypes (IgM, and different isotypes like IgG1, IgG2a, and IgG2b), or the same affinity reagent type but with different target analytes. Different conjugation methods include different stringency of reduction when thiol-reactive chemistry is used to conjugate affinity reagents to mass tags. Different mass tags include different high sensitivity polymers (e.g., having different chelating groups, polymer sizes, polymer shapes, and/or different composition of solubility enhancing groups). For example, a higher stringency conjugation (e.g., reduction) may be used for affinity reagents that present fewer attachment sites (e.g., thiol groups). Kits of the subject application include a plurality of different affinity reagents conjugated to mass tags having different polymeric structures (e.g., in addition to having different labeling atoms).

In certain aspects, different affinity reagents (including different antibody immunoglobulin classes) may be conjugated to different mass tags, or may be conjugated under different conditions to chemically identical or similar mass tags. For example, when thiol-reactive chemistry is used, certain antibodies may respond differently to reduction (e.g., by TCEP). As such, a plurality of affinity reagents may be conjugated to the same mass tag polymer structure (potentially loaded with different isotopes) by different conjugation protocols.

Small moiety affinity reagents that are mass tagged may be purified by methods other than FPLC, such as by spin filtration. In certain aspects, a mass tag (or total amount of mass tags) bound to an affinity reagent may be at least 20%, 30%, 50%, or 80% of the size of the affinity reagent itself, which may allow for spin filtration. In certain aspects, mass tagged antibodies may be purified by spin filtration.

### Cellular Sample

A cellular sample may be any biological sample with intact whole cells. For example, a cellular sample may be a tissue section, such as a tissue section with a thickness of greater than 20 μm, greater than 30 μm, or greater than 50 μm. The tissue section may be fixed such as a formalin-fixed paraffin-embedded (FFPE) tissue section or a tissue section embedded in a matrix such as matrigel. Alternatively, the tissue section may be fresh-frozen, such as a section of a snap frozen tissue. The tissue section may be prepared prior to applying the spatial barcode.

A cellular sample may be a live solid tissue sample, such as an organoid or a tissue biopsy, or an organ or portion thereof harvested from a subject such as a mouse model.

The cellular sample may be fixed, such as by FFPE or embedded in a matrix such as Matrigel (a basement membrane matrix provided by Corning Life Sciences).

The cellular sample may comprise mass cytometry reagents, such as a metal-containing biosensor or metal-containing histochemical compound to the cellular sample (e.g., applied prior to the step of suspending the spatially barcoded cells). The metal may have an atomic mass above 80 amu and may be isotopically enriched.

### Spatial Barcoding

Aspects of the subject application include applying a spatial barcode to a cellular sample prior to performing the above described suspension mass cytometry.

In certain aspects, a method of spatial barcoding for suspension mass cytometry may include one or more of: applying a spatial barcode to a cellular sample such that cells in different locations of the cellular sample are labeled with different combinations or ratios of isotopes (i.e., barcode isotopes), wherein the spatial barcode comprises enriched isotopes having an atomic mass greater than 80 amu; suspending spatially barcoded cells of the cellular sample; staining the suspended cells with mass-tagged affinity reagents, wherein the mass-tagged affinity reagents comprise enriched isotopes having an atomic mass greater than 80 amu and distinct from the atomic mass of the enriched isotopes of the spatial barcode; and analyzing the cells by suspension mass cytometry such that the enriched isotopes of the spatial barcodes and the enriched isotopes of the mass-tagged antibodies are detected on a cell-by-cell basis.

Aspects include applying a spatial barcode to a solid sample comprising whole cells, such as a thick tissue section. After cells are barcoded based on their location in the sample, cells are dissociated, stained with Maxpar conjugated antibodies, and analyzed by suspension mass cytometry. The spatial barcode on each cell can be used to identify where it was in the sample.

Described herein are methods and kits for spatially barcoding a tissue sample so that the tissue can then be dissociated into single cell for analysis on suspension mass cytometry (SMC) instead of IMC. Since the samples are acquired on SMC, sample throughput is increased, there is an increase in signal sensitivity, and there are increased mass tag reagents available for SMC. The spatially barcoded sample can be rendered against an optical image prior to dissociation, allowing the location of the cell to be identified in the original tissue.

This solution could provide a complementary workflow for customers who do not have access to IMC and want to perform high parameter analysis on banked frozen tissue samples. Currently, dissociated tissue samples acquired on SMC can be challenging. Users typically rely on staining fresh tissue which is first mechanically and enzymatically dissociated into single cell for staining and acquisition. Nonetheless, this conventional workflow does not maintain any spatial information regarding the location of the cells analyzed in relation to the original tissue structure. Spatial barcoding could allow SMC customers to be able to analyze tissue section samples while maintaining spatial information. Since this would be acquired in SMC, it would have the advantage of increased throughput, sensitivity, and available mass channels compared to IMC.

Described herein is a means for spatially barcoding a tissue sample in 2 or 3 dimensions to allow for dissociation of single cells and analysis in a suspension mass cytometry workflow (e.g., as opposed to an LA-ICP-MS workflow). The spatial barcode retains spatial information about each cell. This approach has the potential to increase cell throughput 100 fold, increase sensitivity 10 fold (as the entire cell will be analyzed), render cell segmentation unnecessary, and increase the plexity (number of available mass tags) by 50% compared to a traditional LA-ICP-MS IMC workflow (which has fewer mass tags validated).

Ideal sample types would be fresh tissue as opposed to FFPE samples, as FFPE samples would be more difficult to dissociate to single cells. That said, Miltenyi GentleMACS and other sample prep solutions may be suitable for FFPE tissue dissociation.

### Sample Type

One aspect to develop is the method and kit for spatially barcoding a sample. These examples we will assume the sample is a thick (e.g., 50-200 μm thick) fresh or fixed tissue section, and explore 3 possible ways to apply a spatial barcode. This relates to features 1 and 2 in section 5.v) below. In general, the barcode is applied to the sample in a spatially organized manner, and allowed to diffuse into the sample. The rate of diffusion of the barcode across the surface of a sample should be slower than the rate at which the barcode binds or is otherwise retained by cells. Fixing the sample in a matrix (such as Matrigel) may further control this rate of diffusion.

### Spatial Barcode Reagents

A number of reagents (e.g., isotopically enriched monoisotopic metal reagents that bind to cells) may be suitable spatial barcodes.

In certain aspects, the spatial barcode reagents could bind to or embed in the cell surface. For example, lipid-functionalized polymer mass tags could embed in the cell surface. Alternatively, lipid-functionalized oligonucleotides could be embedded in the cell surface in a first step, and could be hybridized to the lipid-oligonucleotides in a second step. Such kinetics for lipid embedding and oligonucleotide hybridization are relatively quick (on the order of minutes). See, for example, MULTI-seq paper published by the Gartner lab for associating lipid-oligonucleotides with cells (McGinnis, C. S., Patterson, D. M., Winkler, J. et al. MULTI-seq: sample multiplexing for single-cell RNA sequencing using lipid-tagged indices. Nat Methods 16, 619-626 (2019)). While antibody staining is slower, a first step of staining cells with an antibody-oligonucleotide conjugate could be followed by a second faster step of hybridizing a spatial barcode of mass tagged oligonucleotides to the antibody-oligonucleotides.

Cells may be patterned to present a molecule, such as biotin/avidin or an oligonucleotide, as an attachment site for the barcode. For example, an antibody mix (e.g., of anti-CD45 and other antibodies to common surface markers) may be applied to the sample in a slow (e.g., >1 hr) incubation step. Streptavidin pre-conjugated to the antibodies may provide an attachment site for a biotinylated metal barcode to attach in a fast (e.g., 2-10 min) incubation step.

Biotin binding to avidin (e.g., neutravidin or streptavidin) may be used to quickly apply a spatial barcode. For example, a biotin (or avidin) could be bound to the cell surface by an antibody or lipid conjugate, after which an avidin (or biotin) functionalized barcoding reagent could be applied to the sample.

Although a mass tagged antibody would be slower to stain the cells, the size of the antibody may result in a slow diffusion through the sample such that mass tagged antibodies could be used to directly apply a spatial barcode. That said, the penetration depth of an antibody stain may be low (e.g., on the order of micrometers) such that a small molecule spatial barcode is preferred.

Alternatively, the spatial barcode reagents may react on or in the cell to covalently bind to the cell. For example, cisplatin or a mass tag functionalized with maleimide for thiol-reactivity (e.g., TeMal, SeMal, or a maleimide functionalized DOTA-metal chelate) could be used as a spatial barcode. See, for example, TeMal publications by the Nitz group, including their 2020 Nature paper to barcoding of whole organoids (Qin, X., Sufi, J., Vlckova, P. et al. Cell-type-specific signaling networks in heterocellular organoids. Nat Methods 17, 335-342 (2020)).

Other organometals or small molecules such as cisplatin may be used in addition to, or as an alternative to, TeMal as described above. Alternatively, a functionalized metal chelate (such as a maleimide-functionalized DOTA-lanthanide chelate) could be used. The current palladium barcode reagent provided by Fluidigm (e.g., in a DMSO solution) could be used, provided the cells are fixed. Another reagent, such as iridium, may also work on fixed and/or permeabilized cells. As with the TeMal example above, at least 2 monoisotopic forms of the barcoding reagent may together provide the spatial barcode such that each location of the sample has a unique combination/ratio of the monoisotopic barcode reagents.

Lipid embedding in membrane, oligonucleotide hybridization to target oligonucleotides attached to the cell surface (e.g., by staining with an antibody-oligonucleotide or lipid-oligonucleotide conjugate), and notably reaction with proteins on or in the cell (e.g., by TeMal, SeMal, cisplatin or maleimide-DOTA) would be expected to barcode the sample in minutes based on the literature; faster than the barcode molecule (e.g., oligonucleotide, lipid or TeMal based) would be expected to diffuse across the entire sample. Attaching a steric group to the barcode reagent and/or embedding the sample in a matrix (e.g., Matrigel) could slow diffusion, while adjusting the conditions such as the pH or temperature may control the rate of binding. As such, barcodes could be applied to discrete locations of the sample.

### Applying Spatial Barcode

The invention may use a small molecule barcoding reagent, such as TeMal, which would diffuse through a thick tissue section (e.g., between 20 μm and 200 μm thick) with whole cells. Below are three distinct examples of how barcodes could be spatially applied to such a sample. Spotting, diffusion and microfluidic delivery could be used alone or in any combination to create a spatial barcode and/or apply it to a cellular sample.

### Spotting

If the barcode can be dried down onto a surface (e.g., applied in a solution which is then evaporated), or if the barcode can be retained by a gel (e.g., a matrix such as a hydrogel, matrigel, polyacrylamide, or gelatin) or adsorbed on a surface such as a film, then a spotting approach may be used. In general, this approach can spot separate mixtures (each having a different combination of amounts of the barcoding isotopes) with 200 μm or less spacing, 100 μm or less spacing, or 50 μm or less spacing. One isotope concentration may be increased in spots along a first dimension (e.g., x-axis), while another isotope concentration may be increased in spots along a second dimension (e.g., y-axis), such that the spot the farthest in the x and y direction has the most of both isotopes. Certain spots may have unique isotopes or combinations thereof to identify the location of the spot. The tissue section may be applied to the surface, wetting the barcode and allowing it to diffuse into the tissue section. Cells between spots may get partial barcoding from each spot, allowing for the cell positions to be identified with better than 100 μm precision (e.g., even if spots were space 100 μm apart).

### Diffusion

Two separate solutions comprising the first and second barcoding isotopes may each be applied to separate edges of a surface (e.g., edges sharing a corner to provide a x and y axis) and allowed to diffuse across the surface. Principles could be similar to thin layer chromatography. Diffusion may occur in the sample, or diffusion could be across the surface of a film that has a coating to adsorb the barcoding reagent (after which the film could be applied to the sample). Alternatively, the two solutions could each be applied directly to a different edge of the tissue section itself at different times. Note that the linear signal provided by CyTOF is an advantage here, as the amount of a particular barcode isotope on a cell could be directly related to its position.

Note that in either the spotting or diffusion example, barcoding reagent could be applied to the surface (e.g., film or gel) shortly before application to the sample, or the solution could be evaporated off, leaving the barcode stationary on the surface. Drying down the barcode or storing the barcode in a non-aqueous solution such as DMSO may reduce is reactivity during storage (e.g. prevent hydrolysis) and allow for a longer shelf life.

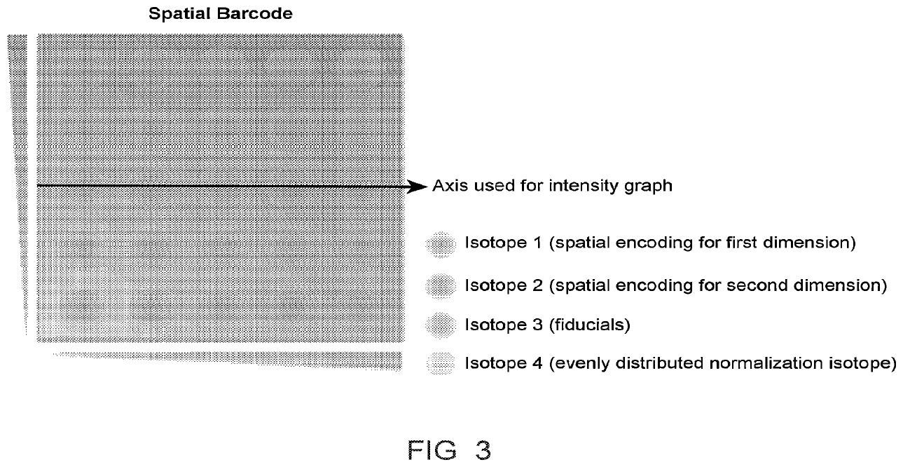

FIG. 3 shows a combination of spotting and diffusion to label a cellular sample directly or prepare a solid support that could then be directly applied to a cellular sample. A first and second isotope may increase along the X and Y axes, another isotope may be patterned in puncti (spots), and another isotope may be uniformly distributed across the sample such that it can be used for normalization (e.g., if different cells demonstrate different uptake of the barcode).

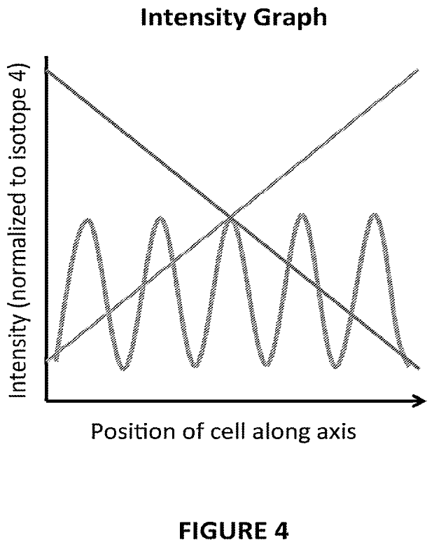

FIG. 4 shows the expected signal intensity for different barcode isotopes depending on the position of a cell along the axis labeled in FIG. 3. After analysis by suspension mass cytometry, these signal intensities for barcode isotopes may be used to identify the location of the cell (e.g., its locations before it was dissociated from tissue).

### Microfluidic Delivery

A barcode may be spatially applied to a sample by microfluidic delivery system. For example, FIG. 2A below from this 2019 Cell paper (Liu et al., “High-Spatial-Resolution Multi-Omics Atlas Sequencing of Mouse Embryos via Deterministic Barcoding in Tissue,” Cell (2020)) shows use of a microfluidic device to apply a barcoding solution along columns and rows. Microfluidic channels can be 10 μm, 25 μm, or 50 μm in width. The bold grid pattern is due to the fact that the barcoding reagents in this paper were with macromolecules (antibodies or oligonucleotides). By design, at least some barcoding reagent would diffuse to the other side of the tissue section before reacting. As such, barcoding reagent of the subject invention would diffuse between the channel markings shown in red and green shown below.

### Analysis

After spatial barcoding, cells can be dissociated from the solid sample (e.g., thick tissue section), stained with mass tagged antibodies, and then analyzed by suspension mass cytometry.

Similar to IMC, the spatial barcoding approach described here may be combined with coregistration of an optical image (e.g., a brightfield image of a histochemical stain of the same or subsequent tissue section). Mass channels outside of the spatial barcode channels may be used for metal containing sensors (e.g., Telox or TePhe), Cell-ID reagents (e.g., cisplatin for viability), RNA detection (e.g., in addition to protein detection), sample barcoding, and any of the current Maxpar antibodies and panels (e.g., cell surface markers and intracellular signaling markers).

The spatial barcode information may be used in a number of ways. For example, cells may still be classified by FCS express or a similar workflow. The spatial barcode may be used to identify a coordinate position of a cell, or a relative position of the cell with respect to other cells. The relative proximity of different cell types to one another may be calculated. A user may define an region of interest (e.g., in a coregistered image obtained before the tissue section was dissociated) and be provided with the cells within that ROI. Bioinformatics may also be used to adjust the location determined for each cell based on the assumption of generally even cell distribution across the sample.

Spatially arranged metal (e.g., metal isotope) barcode that imprints a solid tissue sample (e.g., fresh frozen tissue) upon application.

As illustrated by the three examples provided above, there are a variety of ways to spatially apply a barcode to a tissue sample. Further, there are a number of patterns that offer benefits, such as a gradient of isotopes across different axes and/or puncti (or other fiducials) comprising an isotope or combination of isotopes described above. In one further example, puncti could be alternating (e.g., in a checkerboard pattern switching between two different isotopes). As discussed, an isotope may be applied at even concentration across the sample for normalization of the barcode signal. An isotope could be applied to the opposite face of the sample to allow for better 3D spatial analysis. A stop reagent, such as a thiolated reagent that reacts with TeMal, could be applied in solution above the tissue section to react with any barcoding reagent that escapes the tissue section; this would reduce off target barcoding from reagents that diffused out of tissue and then re-entered the tissue at a different location. Washes may be performed to remove excess barcoding reagent.

Barcodes could comprise a dye or fluorophore, or be in admixture with a dye or fluorophore, so that optical interrogation could identify which parts of the sample received which barcodes. For example, the microfluidic delivery shown in example 3 could apply a fluorophore alongside the barcode, and an optical microscopy image of the barcoded sample could be coregistered with the spatial mass cytometry data to map cells to locations in the image.

TeMal may be an ideal barcode as it is a small molecule (diffuses in tissue), has been shown to label live cells with low toxicity, can be used for live or fixed cells, and has been shown to work for whole organoids. It may be possible to dry down TeMal on a surface or adsorb it to a surface for application to a sample without risk of hydrolysis or oxidation during storage, such as by evaporating a solvent such as toluene. However, if TeMal needs to be stored in a liquid (e.g., in DMSO) for stability, it may be spatially arranged on a surface shortly before application to a sample, or may be directly applied (e.g., in a microfluidic workflow to the sample as described in Example 3). In another alternative, a TeMal derivative comprising a protected maleimide could spatially arranged (e.g., on a gel or film) and, prior to or during application, could be deprotected by a reverse Diels-Alder reaction.

Barcodes will ideally react, or other wised associate with cells, quickly (on the order of minutes) and diffuse deeply into tissue (e.g., diffuse across 100 μm of tissue on the order of minutes, but not diffuse across the entire sample too rapidly). The conditions can be adjusted to change the rate of the barcoding reaction (e.g., the acidity of the solution may affect the rate at which TeMal barcodes cells) or to change the rate of diffusion (e.g., adding as steric group to the barcode reagent could slow its diffusion rate in the sample if needed).

### TeMal Barcoding Reagents

Organotellurim (TeMal) and cisplatin barcoding reagents are described in the context of sample barcoding organoids by Qin, Xiao, et al. (“Cell-type-specific signaling networks in heterocellular organoids.” Nature methods 17.3 (2020): 335-342).

### Additional Aspects for Spatial Barcoding

Additional steps to a spatial barcoding SMC method are described herein. One or more such steps may be included in any method or enabled by any kit of the subject applications.

After dissociation, cells of interest may be enriched. For example, cells MACSselect beads may be used to enrich for cells expressing certain cell types of interest prior to staining or analysis by mass cytometry.

Spatial barcoding could be combined with sample barcoding, such as to barcode different sample types or treatment conditions and/or reduce doublets that would lead to confusing or incorrect spatial barcoding readouts.

Application of biosensors, such as tellurophenes that are enzyme substrates, to the sample prior to suspending the cells, would allow for further interrogation of the tissue microenvironment.

Co-registration with optical microscopy (such as brightfield, fluorescent, confocal, or photoacoustic), optionally in combination with a colormetric histochemical stain and/or fiducials, could allow cells analyzed by SMC to be related to tissue morphology or regions of interest.

2D or 3D coordinates could be provided for cells based on signal from spatial barcode channels and optionally integrated will cell type analysis such as cell gating. Proximity scores between different cell types could be determined. A user may define an ROI and identify cells within that ROI for further analysis.

If some cells or regions of the sample do not have consistent relationship between barcode isotope intensity and location, such cell events may be removed from the SMC dataset (e.g., by removing cell events at edges of sample).

Fiducials or dyes may be used to identify the distribution of barcode isotopes. For example, a dye that is applied alongside an isotope and that has similar diffusion properties through the sample could be used to determine when the spatial barcode has been properly applied to the sample and/or help coregister cell events detected by SMC with an image obtained from the sample.

### Controlling Spatial Barcode Dynamics

Devices, reagents and method steps may further control the application of a spatial barcode to a cellular sample. In general, the spatial barcode may react locally in the cellular sample (e.g., faster than it diffuses homogenously across the entire sample).

Application of chemical gradients is well known in bioengineering. In certain aspects, 2 or more barcode isotopes may diffuse from (or be applied to) the same point or edge but for different times to create gradients across different distances. Alternatively or in addition, the 2 or more barcode isotopes may have different diffusion or reaction rates.

Intensity may be a function of diffusion and reaction (e.g., covalent bonding, affinity binding, or uptake by cells). Diffusion may be controlled by temperature, the medium (e.g., the density of a matrix the sample is embedded in), or by the steric hindrance of the barcode itself (e.g., adding a steric group may reduce diffusion rate). Reaction rate may be controlled by temperature or pH or chemicals or cofactors in admixture with the barcode.

A density gradient or thickness gradient of a gel (e.g., hydrogel, matrigel, polyacrylamide, or gelatin) could be used to control how much of a barcode is retained by the gel, and the gel could then be applied to the cellular sample to deliver a gradient of one or more barcode isotopes.

Passive diffusion or active flow could be used to control a spatial barcode isotope gradient.

A normalization isotope could be applied to the cellular sample before and/or after dissociation (suspension) of cells for SMC steps.

Different barcoding isotopes may be applied to opposite sides of a sample, for example, to provide a 3D distribution of the barcode isotopes.

Spatial barcode solid supports may be a microscope slide such that a sample may be imaged and/or further prepared as the spatial barcode takes place. Such slide may have fiducials for coregistration as discussed herein.

Stop reagents or steps could include an excess of a reagent that reacts with the barcode and/or a change in pH or temperature. A sterically hindered stop reagent could capter barcode exiting tissue (e.g., a steric reagent presenting thiols could capture TeMal exiting tissue).

In certain aspects, at least 90% of the spatial barcode will react with tissue by 5 minutes, 10 minutes, or 20 minutes after application to the cellular sample.

Methods of making a spatial barcode for suspension mass cytometry, may include: distributing spatial barcode isotopes in solution across a solid support; and evaporating the solvent; wherein the spatial barcode isotopes are isotopically enriched and wherein different locations of the solid support comprise different combinations and/or ratios of the spatial barcode isotopes.

In certain aspects, distributing may include spotting spatial barcode isotopes. Distributing may include diffusing one or more spatial barcode isotopes across the solid support

The solvent is an organic solvent having a higher vapor pressure than water at room temperature. For example, the solvent may be toluene.

Aspects also include a cellular sample spatially barcoded by any of the methods or kits described herein.

### Kits

Kits for mass cytometry may include one or more reagents or devices described herein. Any combination of the above described components may be provided in a kit. For example, a spatial barcode kit for suspension mass cytometry, may include: a plurality of barcodes comprising enriched isotopes having an atomic mass greater than 80 amu; and a device configured to apply the barcodes to a cellular sample in a spatially arranged manner.

Wherein the device may include a solid support such as a film or gel (e.g., matrix such as a hydrogel, polyacrylamide or gelatin). Spatial barcode isotopes may be patterned across at least a portion of the solid support such that each location is uniquely barcoded.

Wherein the device may include a microfluidic device, e.g., comprising channels configured to deliver a different combination and/or ration of barcodes to different locations of a sample

Barcodes comprising different enriched isotopes may be packaged separately, in unique combinations of enriched isotopes, or in unique ratios of enriched isotopes.

Wherein the barcodes may be dried down or may be suspended in a solvent such as DMSO.

Any of the methods described herein may be totally or partially performed with a computer system including one or more processors, which can be configured to perform the steps. Thus, embodiments can be directed to computer systems configured to perform the steps of any of the methods described herein, potentially with different components performing a respective step or a respective group of steps. Although presented as numbered steps, steps of methods herein can be performed at a same time or at different times or in a different order that is logically possible. Additionally, portions of these steps may be used with portions of other steps from other methods. Also, all or portions of a step may be optional. Additionally, any of the steps of any of the methods can be performed with modules, units, circuits, or other means of a system for performing these steps.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure.

The above description of example embodiments of the present disclosure has been presented for the purposes of illustration and description and are set forth so as to provide those of ordinary skill in the art with a complete disclosure and description of how to make and use embodiments of the present disclosure. It is not intended to be exhaustive or to limit the disclosure to the precise form described nor are they intended to represent that the experiments are all or the only experiments performed. Although the disclosure has been described in some detail by way of illustration and example for purposes of clarity of understanding, it is readily apparent to those of ordinary skill in the art in light of the teachings of this disclosure that certain changes and modifications may be made thereto without departing from the spirit or scope of the appended claims.

Accordingly, the preceding merely illustrates the principles of the invention. It will be appreciated that those skilled in the art will be able to devise various arrangements which, although not explicitly described or shown herein, embody the principles of the invention and are included within its spirit and scope. Furthermore, all examples and conditional language recited herein are principally intended to aid the reader in understanding the principles of the disclosure being without limitation to such specifically recited examples and conditions. Moreover, all statements herein reciting principles, aspects, and embodiments of the invention as well as specific examples thereof, are intended to encompass both structural and functional equivalents thereof. Additionally, it is intended that such equivalents include both currently known equivalents and equivalents developed in the future, i.e., any elements developed that perform the same function, regardless of structure. The scope of the present invention, therefore, is not intended to be limited to the exemplary embodiments shown and described herein. Rather, the scope and spirit of present invention is embodied by the appended claims.

A recitation of “a”, “an” or “the” is intended to mean “one or more” unless specifically indicated to the contrary. The use of “or” is intended to mean an “inclusive or,” and not an “exclusive or” unless specifically indicated to the contrary. Reference to a “first” component does not necessarily require that a second component be provided. Moreover, reference to a “first” or a “second” component does not limit the referenced component to a particular location unless expressly stated. The term “based on” is intended to mean “based at least in part on.”

The claims may be drafted to exclude any element which may be optional. As such, this statement is intended to serve as antecedent basis for use of such exclusive terminology as “solely”, “only”, and the like in connection with the recitation of claim elements, or the use of a “negative” limitation.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within embodiments of the present disclosure. The upper and lower limits of these smaller ranges may independently be included or excluded in the range, and each range where either, neither, or both limits are included in the smaller ranges is also encompassed within the present disclosure, subject to any specifically excluded limit in the stated range. Where the stated range includes one or both of the limits, ranges excluding either or both of those included limits are also included in the present disclosure.

All patents, patent applications, publications, and descriptions mentioned herein are hereby incorporated by reference in their entirety for all purposes as if each individual publication or patent were specifically and individually indicated to be incorporated by reference and are incorporated herein by reference to disclose and describe the methods and/or materials in connection with which the publications are cited. None is admitted to be prior art.

1. A method of spatial barcoding for suspension mass cytometry, comprising:
a) applying a spatial barcode to a cellular sample such that cells in different locations of the cellular sample are labeled with different combinations or ratios of isotopes, wherein the spatial barcode comprises enriched isotopes having an atomic mass greater than 80 amu;
b) suspending spatially barcoded cells of the cellular sample;
c) staining the suspended cells with mass-tagged affinity reagents, wherein the mass-tagged affinity reagents comprise enriched isotopes having an atomic mass greater than 80 amu and distinct from the atomic mass of the enriched isotopes of the spatial barcode; and
d) analyzing the cells by suspension mass cytometry such that the enriched isotopes of the spatial barcodes and the enriched isotopes of the mass-tagged affinity reagents are detected on a cell-by-cell basis.

2. The method of claim 1, wherein the cellular sample is a tissue section.

3. The method of claim 2, wherein the tissue section has a thickness of greater than 20 microns.

4. The method of claim 2, wherein the tissue section is a formalin-fixed paraffin-embedded (FFPE) tissue section.

5. The method of claim 1, wherein the cellular sample is a live solid tissue sample.

6. The method of claim 1, wherein the cellular sample is a fresh-frozen solid tissue sample.

7. The method of claim 1, wherein the cellular sample is an organoid.

8. The method of claim 1, wherein the cellular sample is a solid tissue biopsy.

9. The method of claim 1, wherein the cellular sample is embedded in a protein matrix.

10. The method of claim 1, further comprising applying a metal-containing biosensor or metal-containing histochemical compound to the cellular sample prior to the step of suspending the spatially barcoded cells.

11. The method of claim 1, wherein the cells at different locations comprise different combinations of isotopes.

12. The method of claim 1, wherein the cells at different locations comprise different ratios of isotopes.

13. The method of claim 1, wherein the spatial barcode is a solid support comprising a distribution of spatial barcode isotopes, wherein the spatial barcode isotopes are patterned across at least a portion of the solid support such that each location is uniquely barcoded.

14. The method of claim 13, wherein the solid support is a film or matrix.

15. The method of claim 1, wherein a first barcode isotope increases along a first spatial dimension in the cellular sample.

16. The method of claim 17, wherein a second barcode isotope increases along a second spatial dimension in the cellular sample.

17. The method of claim 18, wherein a third barcode isotope is spotted at multiple locations in the cellular sample.

18. The method of claim 1, wherein at least 2 barcode isotopes have a unique spatial distribution.

19. The method of claim 1, wherein a normalization barcode isotope is evenly distributed across at least a portion of the cellular sample.

20. The method of claim 1, wherein the spatial barcode is arranged on a solid support that is applied to the cellular sample.

21. The method of claim 1, wherein the spatial barcode is applied to the cellular sample by a microfluidic device.

22. The method of claim 21, wherein the microfluidic device comprises channels configured to deliver a different combination or ratio of barcode isotopes to different locations of a sample.

23. The method of claim 1, wherein applying the spatial barcode comprises diffusing at least one isotope across the cellular sample.

24. The method of claim 1, wherein applying the spatial barcode comprises diffusing the spatial barcode isotopes across the cellular sample in different directions.

25. The method of claim 1, wherein applying the spatial barcode comprises contacting the cellular sample with a solid support and then applying a solution of one or more spatial barcode isotopes to the opposite side of the solid support from the cellular sample.

26. The method of claim 25, wherein the solid support is a gel or a porous substrate that provides a gradient of permeability.

27. The method of claim 1, wherein the spatial barcode comprises a solid support spotted with different combinations or ratios of barcode isotopes.

28. The method of claim 1, further comprising applying an even distribution of a normalization isotope to the cellular sample before and/or after the step of suspending the cells.

29. The method of claim 1, wherein the spatial barcode comprises a thiol-reactive moiety.

30-32. (canceled)

33. The method of claim 1, wherein the spatial barcode comprises a dye or fluorophore that indicates the distribution of the spatial barcode isotopes.

34-38. (canceled)

39. The method of claim 138, wherein analyzing the cells by suspension mass cytometry comprises introducing the cells to ICP-TOF-MS.

40-42. (canceled)

43. The method of claim 1, further comprising analyzing the 3D spatial distribution of cells based on the enriched isotopes of the spatial barcodes detected for individual cells

44-49. (canceled)
